(19) United States
(12) Patent Application Publication
(10) Pub. No.: US 2025/0359774 A1
(43) Pub. Date: Nov. 27, 2025

(54) DEVICES, METHODS, AND SYSTEMS FOR COLLECTING AQUATIC FIELD METABOLIC RATE DATA

(71) Applicants: Stephanie Marie Dohner, Newark, DE (US); Aaron Ball Carlisle, Newark, DE (US); Jonathan Hirsch Cohen, Newark, DE (US); Arthur C. Trembanis, Newark, DE (US); Andrea Leontiou, Newark, DE (US)

(72) Inventors: Stephanie Marie Dohner, Newark, DE (US); Aaron Ball Carlisle, Newark, DE (US); Jonathan Hirsch Cohen, Newark, DE (US); Arthur C. Trembanis, Newark, DE (US); Andrea Leontiou, Newark, DE (US)

(73) Assignee: University of Delaware, Newark, DE (US)

(21) Appl. No.: 18/874,111

(22) PCT Filed: Jun. 13, 2023
(86) PCT No.: PCT/US2023/025157

§ 371 Date: Dec. 12, 2024

Related U.S. Application Data
(60) Provisional application No. 63/351,472, filed on Jun. 13, 2022.

(51) Int. Cl. A61B 5/083; A61B 5/11

(52) U.S. Cl. CPC A61B 5/0833; A61B 5/1113; A61B 2503/40; A61B 2560/0242

(57) ABSTRACT

A device collects aquatic field metabolic rate data related to a water-breathing animal having at least one gill opening. The device has at least one oxygen probe, which measures and records oxygen consumption or oxygen extraction data at the at least one gill opening. The device is configured to collect metabolic data when the animal is in a natural environment. A method for determining a metabolic rate of the animal located in a water environment includes measuring metabolic rate data via at least one gill oxygen probe which is inserted into the at least one gill opening; measuring ambient data in the water environment via at least one ambient oxygen probe; and estimating a whole animal metabolic rate.

### CROSS-REFERENCE TO RELATED APPLICATIONS

This application claims priority from U.S. Provisional Application Ser. No. 63/351,472, titled “RemO2ra Aquatic Metabolic Biologging Tag,” filed Jun. 13, 2022, the entirety of which is incorporated herein by reference.

### BACKGROUND OF THE INVENTION

Metabolic rate is a measure of the energetic cost of life and is integral to understanding an animal's life history, behavior, and trophic impact, and, by extension, how the population and ecosystem function. For example, field metabolic rate (FMR) represents the animal's in situ dynamic energetic response to its environment, activity, and physiological conditions. Aspects of metabolic rate, such as standard metabolic rate (SMR), maximum metabolic rate (MMR), specific dynamic action (SDA), and aerobic scope (AS), are incorporated into the FMR. Due to both their size and accessibility, relatively few shark species' metabolic rates have been measured. In addition, fish have lower metabolic and heat production rates compared to mammals, which makes adequately sensitive measurements related to determining metabolic rate difficult.

Conventional methods for determining metabolic rate in water breathing marine animals are typically constrained to a lab or semi-captive natural settings. For many larger species, metabolic rates cannot be estimated in either setting, due to logistical and size constraints, making it difficult to study larger animals, such as elasmobranchs (sharks, rays, skates, sawfish, etc.). Said conventional methods for determining adult shark's metabolic rates, such as FMR, include respirometry systems, which fall into one of two categories: swim tunnel or static. Swim tunnel styles use a propeller to generate a current that the fish must swim against to stay in position, while a static respirometer has the fish swimming as it would naturally and volitionally. However, there are inconsistencies between methods, in that studies have found differing metabolic rate measurements, dependent on the system used.

Still further, laboratory studies cannot completely replicate environmental conditions, behavior, and activity. Additionally, conventional methods require lab validation, which is typically difficult for lager animals. Accordingly, metabolic rates of all but a few shark species have been understudied, and the ones that have been studied may not be accurate representations of a shark's metabolic rate in the wild or natural environment.

Thus, it is of interest to develop improvements in devices, methods, and systems concerning collection of metabolic rate data, such as for larger shark species in the wild, particularly without requiring or solely relying on respirometry or preliminary laboratory estimates of metabolic rates.

### SUMMARY OF THE INVENTION

The drawbacks of conventional methods for determining metabolic rate of water breathing marine animals are addressed in many respects by devices, methods, and systems in accordance with the invention.

One aspect of the invention comprises a device configured to collect aquatic field metabolic rate data related to a water-breathing animal having at least one gill opening. The device has a plurality of sensors. The plurality of sensors includes at least one oxygen probe configured to measure oxygen consumption or oxygen extraction data at the at least one gill opening. The device has a controller coupled to the plurality of sensors. The controller is configured to perform one or more of the following operations in accordance with instructions stored in a digital memory: (i) control one or more control settings of the plurality of sensors, (ii) process and record metabolic rate data collected by the plurality of sensors, and (iii) transmit metabolic rate data collected by the plurality of sensors to an external device. The device is configured to collect metabolic rate data when the animal is in a natural environment.

Another aspect of the invention comprises a method for determining a metabolic rate of a water-breathing animal having at least one gill and located in a water environment. The method comprises inserting a plurality of sensors into the at least one gill opening of the animal. The plurality of sensors include at least one gill exhalant oxygen probe. The method includes measuring metabolic rate data of the animal via the at least one gill exhalant oxygen probe. The method also comprises measuring ambient data in the water environment via at least one ambient oxygen probe and at least one ambient temperature probe; and estimating a whole animal metabolic rate from one or more of the collected metabolic rate data and ambient.

### BRIEF DESCRIPTION OF THE DRAWINGS

The invention is best understood from the following detailed description when read in connection with the accompanying drawings, with like elements having the same reference numerals. When a plurality of similar elements are present, a single reference numeral may be assigned to the plurality of similar elements with a small letter designation referring to specific elements. When referring to the elements collectively or to a non-specific one or more of the elements, the small letter designation may be dropped. This emphasizes that according to common practice, the various features of the drawings are not drawn to scale unless otherwise indicated. On the contrary, the dimensions of the various features may be expanded or reduced for clarity. Included in the drawings are the following figures:

FIG. 1 depicts existing devices for determining animal behavior and ambient conditions in water-breathing animals in accordance with prior art;

FIG. 2 depicts a method for determining a metabolic rate of a water-breathing animal in accordance with an exemplary embodiment of the invention;

FIGS. 3A-3C depict a device for collecting aquatic field metabolic rate data in aquatic animals in accordance with an exemplary embodiment of the invention;

FIG. 4 depicts a shark in a static respirometry system in accordance with clinical testing of the device of FIGS. 3A-3C and the method of FIG. 2;

FIG. 5 depicts a schematic placement of components of the device of FIG. 3A in a swim tunnel respirometry system in accordance with clinical testing of the device of FIGS. 3A-3C and the method of FIG. 2;

FIG. 6A depicts a graph showing a comparison of oxygen extraction at each gill in accordance with clinical testing of the device of FIGS. 3A-3C and the method of FIG. 2;

FIG. 6B depicts a graph showing a flow rate of water leaving each gill in accordance with clinical testing of the device of FIGS. 3A-3C and the method of FIG. 2;

FIG. 6C depicts a graph showing an area of each gill opening in accordance with clinical testing of the device of FIGS. 3A-3C and the method of FIG. 2;

FIG. 6D depicts a graph showing mass specific oxygen consumption ±standard deviation (D) for each of the Sandbar Sharks (Light=N, Mid=P, Dark=Y) already sampled (n=3);

FIG. 7 depicts a graph showing a comparison calculated full body metabolism from single gill measurements for each individual Sandbar Shark (A=N, B=P, C=Y) sampled (n=3) in accordance with clinical testing of the device of FIGS. 3A-3C and the method of FIG. 2;

FIG. 8 depicts a graph showing dissolved oxygen (DO) percent saturation and temperature during and after bubbling with nitrogen in accordance with clinical testing of the device of FIGS. 3A-3C and the method of FIG. 2;

FIG. 9A depicts a graph showing a comparison of oxygen extraction at each gill in accordance with clinical testing of the device of FIGS. 3A-3C and the method of FIG. 2;

FIG. 9B depicts a graph showing a comparison of flow rate of water leaving each gill in accordance with clinical testing of the device of FIGS. 3A-3C and the method of FIG. 2;

FIG. 9C depicts a graph showing a comparison of area of each gill opening in accordance with clinical testing of the device of FIGS. 3A-3C and the method of FIG. 2;

FIG. 9D depicts a graph showing a comparison of mass specific oxygen consumption ±standard deviation (D) for each of the Sandbar Sharks (Grey) and Smooth Dogfish (Blue) already sampled (n=6) in accordance with clinical testing of the device of FIGS. 3A-3C and the method of FIG. 2;

FIG. 10 depicts a conceptual diagram showing clinical testing of the device of FIGS. 3A-3C and the method of FIG. 2; and

FIG. 11 depicts a graph showing a comparison of average metabolic rates calculated in accordance with clinical testing of the device of FIGS. 3A-3C and the method of FIG. 2 and average metabolic rates calculated in accordance with conventional respirometry systems and methods.

### DETAILED DESCRIPTION OF THE INVENTION

Aspects of this invention relate to devices, methods, and systems for collecting aquatic field metabolic rate data, and more particularly, to devices, methods, and systems for collecting aquatic field metabolic rate data of sharks in a natural environment.

Although the invention is illustrated and described herein with reference to specific embodiments, the invention is not intended to be limited to the details shown. Rather, various modifications may be made in the details within the scope and range of equivalents of the claims and without departing from the invention.

Additionally, various forms and embodiments of the invention are illustrated in the figures. It will be appreciated that the combination and arrangement of some or all features of any of the embodiments with other embodiments is specifically contemplated herein. Accordingly, this detailed disclosure expressly includes the specific embodiments illustrated herein, combinations and sub-combinations of features of the illustrated embodiments, and variations of the illustrated embodiments.

Aspects of the invention are described herein with reference to sharks. However, it will be understood by one of ordinary skill in the art that the exemplary devices, methods, and systems described herein are not limited to specific aquatic animals or specific species thereof. Other types of marine or aquatic field animals and other species thereof suitable for use with the disclosed devices, methods, and systems will be known to one of ordinary skill in the art from the description herein.

As used herein and throughout the specification, the terms “metabolic rate,” “metabolic rate data,” and “aquatic field metabolic rate data” are intended to encompass physiological characteristics of the subject animal. In particular, one skilled in the art would understand from the description herein that metabolic rate data includes the way the subject animal gains and subsequently uses energy. Specifically, the terms are intended to encompass data related to different aspects of metabolic rate that can be observed and determined (e.g. in a laboratory setting, in a non-captive and non-laboratory setting, etc.) in aquatic animals, including standard metabolic rate (SMR), maximum metabolic rate (MMR), specific dynamic action (SDA), and aerobic scope (AS). SMR is the basic rate of metabolism, and refers to the minimal amount of energy required to maintain a fish that has not eaten and is at rest. On the other hand, MMR is the metabolic rate of an animal after it has reached its highest possible rate of aerobic activity. Aerobic scope, an indicator of an animal's excess metabolic capacity at a given temperature, is the difference between MMR and SMR. SDA is the increase in metabolic cost of digestion and assimilation of food, and is the increase in metabolic rate that occurs after eating. Further, the terms include FMR, which represents the subject animal's in situ dynamic energetic response to its environment, activity, and physiological conditions.

Additionally, one skilled in the art would understand that from the description herein that “metabolic rate data” include characteristics that are at least within a known or expected range of values, which may be actual values as measured, or expected ranges based upon length, weight, temperature, and other physiological factors of the subject animal. The scaling of metabolic rate with body size and temperature has been used to predict ecological roles and life history traits (i.e. the metabolic theory of ecology) across taxa, and has led to general understanding that a decrease in mass specific respiration rate occurs with increased size. According to the metabolic level boundary hypothesis (MLB), volume and surface area act as boundaries—due to the constraints that structural area imposes on resources fluxes and tissue maintenance costs respectively—for the metabolic scaling component, which changes not just with size, but with activity level and ecology as well. As such, once temperature and body size have been accounted for, metabolic rate—usually SMR—can be related directly to a shark's lifestyle (i.e. more sedentary or more active, buccal pumping or obligate ram ventilating). Fish species with higher activity levels tend to have higher growth performance, and a higher metabolic rate for their body mass. In a similar vein, buccal pumping species of sharks are observed to have lower metabolic rates, than obligate ram ventilators, likely due to the increased energetic cost of constant movement and the larger gill surface area required by a higher activity level.

As used herein and throughout the specification, the term “natural environment” refers to, for example, a subject animal's habitat or environment where it is natural for the subject animal to survive and reproduce. The term is intended to encompass physical, ecological, and biotic factors intended to support the survival and reproduction of the subject anima. For example, the natural environment of sharks can be characterized as an aquatic field (e.g. ocean, lakes, rivers, or other bodies of water). Additionally, the term “natural environment” may encompass non-laboratory setting, non-captive setting, semi-captive setting, or combinations thereof.

In addition, as used herein and throughout the specification, the term “exhalant water” refers to deoxygenated water leaving the gills of the subject water-breathing animal.

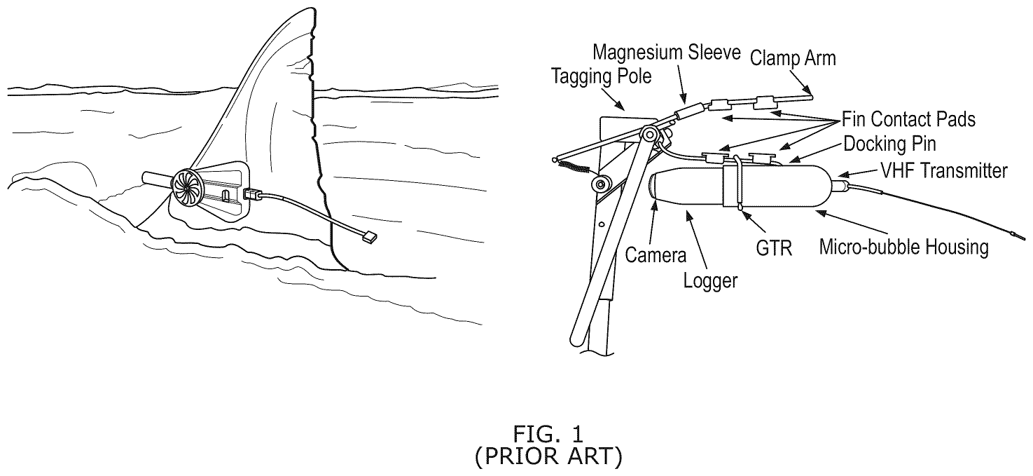

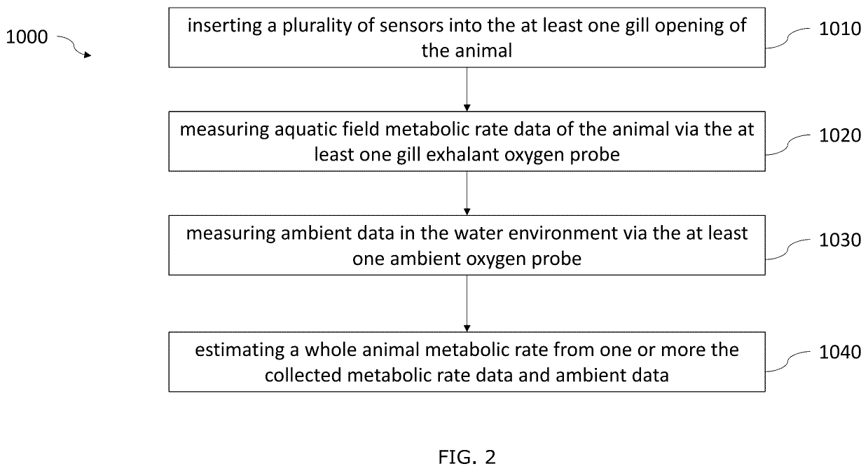

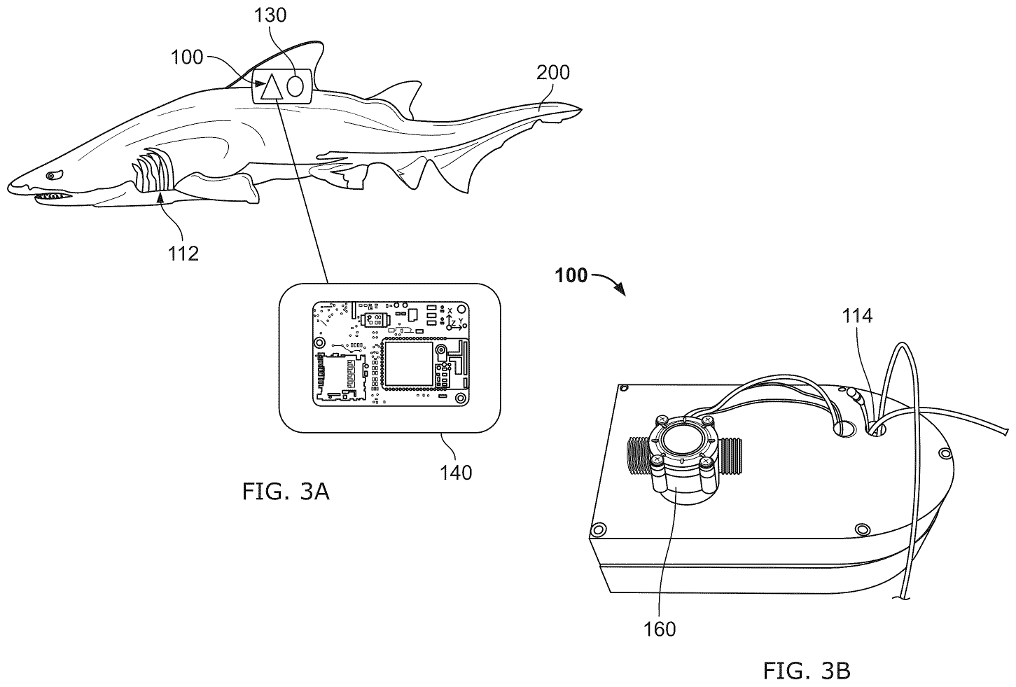

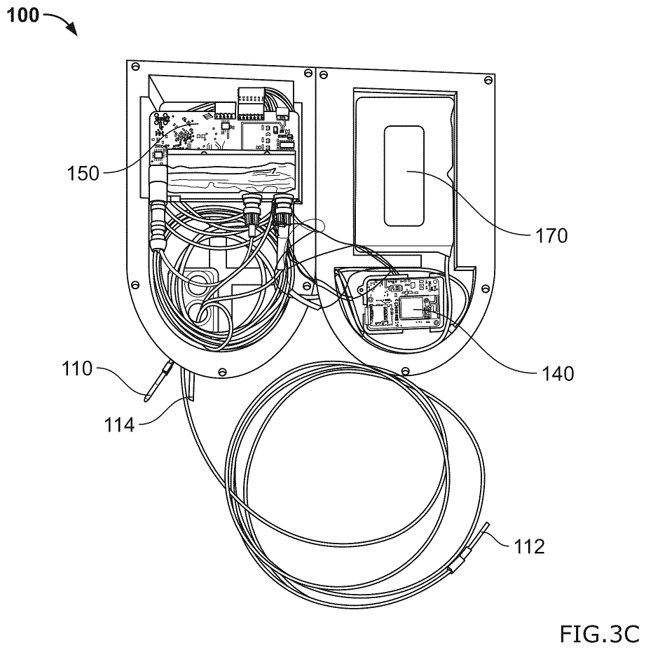

With reference to the drawings, FIG. 1 are images of examples of prior art devices for use in determining animal behavior and ambient conditions of the subject animal (e.g. shark), such as existing Customized Animal Tracking Solutions (CATS) biologging devices, as manufactured and designed by Customized Animal Tracking Solutions Pty. Ltd. Of Queensland, Australia. FIG. 2 depicts an exemplary method 1000 for determining a metabolic rate of a water-breathing animal having at least one set of gills and located in a water environment, in accordance with aspects of the present invention. In one example, the water environment is a tank sealed with a fiberglass lid. Likewise, FIGS. 3A-3C depict an exemplary device 100 configured to collect aquatic field metabolic rate data related to the water-breathing animal having the gills, such as shark 200.

In an exemplary embodiment, as shown in FIG. 2, method 1000 includes one or more steps including inserting a plurality of sensors into at least one gill opening of the animal; measuring aquatic field metabolic rate data of the animal; measuring ambient data in the water environment; and estimating a whole animal metabolic rate from one or more of the collected metabolic rate data and ambient data. Additional details of method 1000 are set forth below with respect to the elements of device 100.

Referring to FIGS. 3A-3C, a device 100 configured to collect aquatic field metabolic rate data related to the water-breathing animal having the gills (e.g. a shark 200) is provided. Additionally, or optionally, one or more components of the device 100 is releasably attached to the shark's 200 dorsal fin (FIG. 3A) or pectoral fin(s). In particular, device 100 may be attached using monofilament and a galvanic timed-release mechanism. In this way, the galvanic timed-release mechanism permits eventual corrosion and subsequent release of one or more components of the device 100 after a predetermined duration (e.g. number of days). Upon corrosion and release of the one or more components of the device 100, said component(s) will float to the surface of the water environment and its location will be transmitted to an external device (e.g. by a controller, as discussed below), thereby allowing for their recovery.

In an exemplary embodiment, device 100 comprises a plurality of sensors. Coupled to the plurality of sensors is a controller 150 (FIG. 3C). The controller 150 is configured to perform one or more of the following operations in accordance with instructions stored in a digital memory: (i) control one or more control settings of the plurality of sensors, (ii) process and record metabolic rate data collected by the plurality of sensors, and (iii) transmit metabolic rate data collected by the plurality of sensors to an external device (e.g. via various communication interfaces known to one skilled in the art). The transmitted metabolic rate data may be real-time data, logged/archival data, or combinations thereof. Additionally, or optionally, the device 100 is configured to collect metabolic rate data when the animal 200 is in a natural environment.

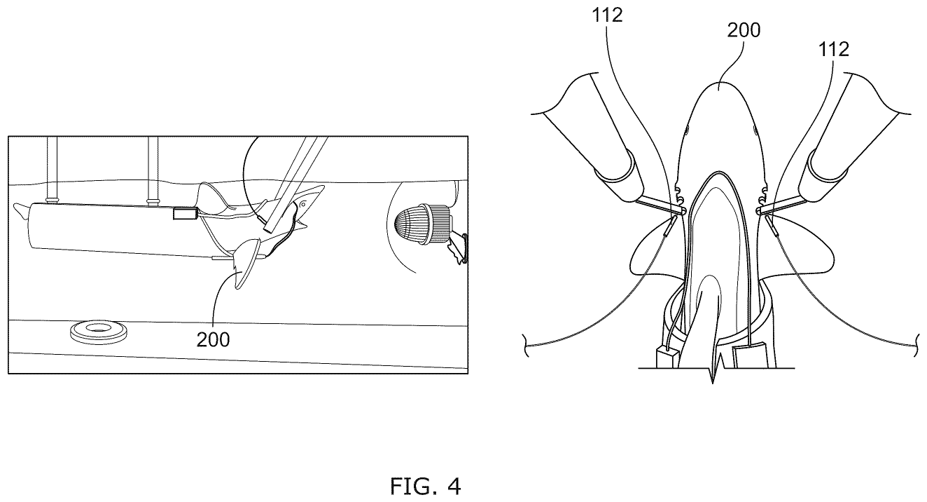

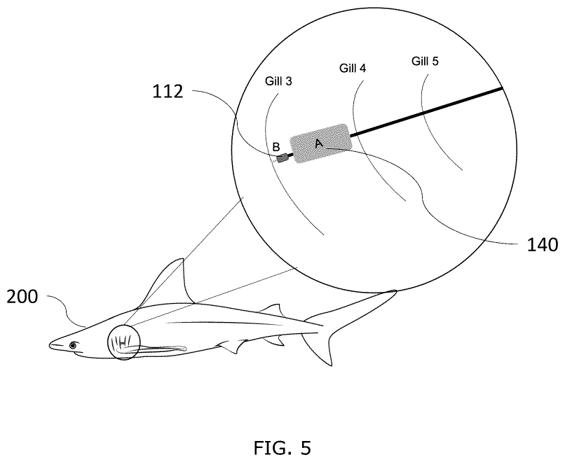

In step 1010, a plurality of sensors is inserted into at least one set of gill openings or at least one gill opening of a water-breathing animal. In an exemplary embodiment, as shown in FIGS. 3A-3C, the plurality of sensors comprises at least one oxygen probe 112. The plurality of sensors is configured to be inserted into at least one set of gills of a water-breathing animal, such as shark 200. As is shown in the art, fish typically have two sets of gills, one on each side of their bodies. Each set of gills may include one gill opening and one or more gill structures (e.g. gill filament) inside each gill opening, wherein the fish takes in water through its mouth, the water passes over the gill structures, and the water exits the fish's body through the gill openings. In another exemplary embodiment, the plurality of sensors is inserted into a respective set of gill openings of the at least the second and/or third gills of the shark 200 (as illustrated in FIG. 4, for example). In another exemplary embodiment, the plurality of sensors is inserted into a respective gill opening of at least the second and/or third gill along one side of a body of the shark 200 (as illustrated in FIG. 5, for example), such that the plurality of sensors is positioned near or adjacent at least the gill filaments over or through which water passes over to facilitate respiration of a water-breathing animal. Additionally, or optionally, the plurality of sensors is inserted up to 5 mm into the gill opening of the third gill.

In step 1020, aquatic field metabolic rate data of the animal via the oxygen probe is measured. Optionally, device 100 includes a temperature probe configured to measure at least a temperature of the animal 200.

In an exemplary embodiment, the oxygen probe comprises at least one gill exhalant oxygen probe 112. In one example, method 100 includes providing a tube 140 sutured to an area behind the measured gill of the animal 200, such that step 1010 includes inserting at least the gill exhalant oxygen probe 112 into or through the sutured tube 140. The gill exhalant oxygen probe 112 is configured to measure oxygen consumption or oxygen extraction data at the gill. In one example, the gill exhalant oxygen probe 112 includes an optical oxygen meter, such as a PyroScience Firesting two channel optical multi-analyte meter with two PyroScience robust oxygen probes, as manufactured and designed by PyroScience GmbH of Aachen, Germany. Further, the gill exhalant oxygen probe 112 is configured to measure dissolved oxygen levels in exhalant water. Additionally, or optionally, the gill exhalant oxygen probe 112 is configured to measure dissolved oxygen levels in exhalant water for a predetermined duration (e.g. 60 seconds) or frequency (e.g. every 60 seconds). In one example, the oxygen consumption or oxygen extraction data comprises incurrent oxygen concentration (CᵢO₂) and excurrent oxygen concentration (CₑO₂) at the gill opening.

In an exemplary embodiment, the plurality of sensors may include at least one ambient oxygen probe 110 and at least one ambient temperature probe 114. The ambient oxygen probe 110 and the ambient temperature probe 114 are submerged in the water environment. In step 1030, the ambient oxygen probe 110 is configured to measure dissolved oxygen levels or (% oxygen saturation) in ambient water in the water environment. In an exemplary embodiment, the measured dissolved oxygen levels in ambient water is comparable to dissolved oxygen levels of inhalant water (e.g. inhalant DO), such as for the purposes of calculating whole organism metabolic rate (explained below). Similarly, the ambient temperature probe 114 is configured to measure temperature data of ambient water in the water environment. In an exemplary embodiment, the temperature of ambient water is comparable to the temperature of the water entering and exiting the at least one gill of shark 200. In one example, the temperature probe 114 includes a Pt100 temperature probe (platinum-based sensor with a resistance of 100Ω at 0° C.).

In an exemplary embodiment, device 100 further comprises a locating device 130. The locating device 130 is configured to be releasably attached to a dorsal fin (FIG. 3A) or pectoral fin of the animal 200. The locating device 130 is configured to track motion or location of the animal 200 or the device 100. Additionally, or optionally, the locating device 130 is configured to transmit, via various communication interfaces known to one skilled in the art, the location of the animal 200 or device 100 to the external device (not shown). Additionally, or optionally, device 100 includes a tri-axial accelerometer 140 (FIG. 3A) configured to collect, record, and/or transmit accelerometry data (e.g. swimming speed) related to motion of the animal 200. In one example, the accelerometer is a Next Generation Inertial Measurement Unit (NGIMU), as designed and manufactured by x-io Technologies Limited of Bristol, UK. Still further, the plurality of sensors comprises at least one sensor (e.g. flow meter 160) configured to measure a flow rate (FR) of water through the respective gill of the animal 200. Additionally, or optionally, as best illustrated in FIGS. 3B-3C, device 100 includes one or more of: a salinity probe configured to measure and record total dissolved salt content in a sample (e.g. the water environment, etc.); a pressure sensor configured to measure depth (e.g. for determining type or location of the subject's water environment, etc.); and a power source (e.g. battery) 170 connected to one or more components of device 100, such as controller 150 and accelerometer 140.

Based on one or more of the collected metabolic rate data and ambient data, a whole animal metabolic rate is estimated in step 1040. In an exemplary embodiment, step 1040 comprises calculating a rate of oxygen consumption over a predetermined duration by the gill (MO₂), which can be expressed by the following equation:

$${MO}_{2} = ( inhalant DO - exhalant DO ) FR$$

MO₂ is the rate of oxygen consumption over a predetermined duration by the at least one gill, inhalant DO is incurrent dissolved oxygen (DO) concentration, exhalant DO is excurrent dissolved oxygen (DO) concentration, and FR is the flowrate of water.

In another exemplary embodiment, step 1040 comprises calculating a rate of oxygen consumption over a predetermined duration by the gill (O₂), which can be expressed by the following equation:

$${O}_{2} = ( {C}_{i} {O}_{2} - {C}_{e} {O}_{2} ) FR$$

In one example, the whole animal metabolic rate is estimated by calculating O₂ using the sum of the detected FR values for a number of gills and multiplying the O₂ value by a relevant factor two (to account for a measurement taken on the number of gills along one side of the subject animal's body is comparable to a measurement that would be taken on the same number of gills along the other side of the subject animal's body). In some embodiments, the subject information is measured at more than one gill to provide a more complete set of measurements to take into account differences between gills and/or measurement systems, and to provide redundancy in the event of sensor failure. In another example, the whole animal metabolic rate is estimated by calculating an average FR and calculating the respective O₂ using the average FR. In still another example, the whole animal metabolic rate further is estimated by calculating an average FR, an average CᵢO₂, and an average CₑO₂, then calculating the respective O₂ using the average values of FR, CᵢO₂, and CₑO₂.

### Example

The co-inventors assessed the exemplary devices, methods, and systems as disclosed herein in a laboratory or semi-captive setting, to validate feasibility and functionality of the components of the subject devices, methods, and systems when used in a natural environment of the animal (e.g. in its natural habitat or environment, in the wild, etc.), as well as verified any updates or improvements made. The prototype devices, methods, and systems were subjected to various tests as detailed herein.

### Example One: Method to Estimate Whole Animal Metabolic Rate from Point Measurements of Gill Exhalant for Sandbar Sharks

Multiple methods were used to determine metabolic rate through oxygen consumption as well as oxygen extraction at the gills for Sandbar Sharks (Carcharhinus plumbeus). This objective, and the others in this study, will use oxygen content in gill exhalant compared to that in the ambient water as the method to determine MO₂. This will help determine if it is possible to use a point reading at an individual gill to determine a full body metabolic rate for an individual animal. Specifically, this objective included the following steps:
1. Determine oxygen extraction and consumption at each gill.
2. Compare oxygen consumption at the gill to whole body consumption in a sealed system.
3. Determine oxygen consumption in the respirometer using the inventive device or components thereof and inventive method or specific steps thereof.

### Hypothesis

Point measurements of oxygen consumption at the gills can be used to reliably estimate a whole animal metabolic rate.

### Methods

### Subject Acquisition & Containment

During the spring, summer and fall of 2021 and 2022, young of the year (YOY) Sandbar Sharks were collected by handline from Delaware Bay. During the trial period, the captured subjects were kept in an indoor 1800 L recirculating seawater tank, connected to a laboratory seawater system. Subject sharks were fed a diet of squid and Atlantic Croaker (Micropogonias undulatus). While in the holding tank, subject sharks in the tank are kept on a 15:9 hour light:dark schedule.

### Individual Gill Measurements of Oxygen Extraction

Referring to FIG. 4, individual oxygen extraction measurements were taken at each set of gills for six Sandbar Sharks (Carcharhinus plumbeus) while they were sedated in a static respirometry system. Each shark was dosed with MS-222 (Tricaine-S) and suspended in a PVC cradle system in a 195 L tank. A powerhead placed 13 cm in front of the shark created a “flow” through its open mouth and gills at a velocity of 0.16 m/s. A prototype of an inventive oxygen probe and/or temperature probe, such as an oxygen probe as manufactured and designed by Pyroscience of Aachen, Germany, was inserted into each gill. Measurements of the dissolved oxygen levels in exhalant water (% air saturation) were collected for a predetermined duration, such as one minute. Another inventive oxygen probe and temperature probe was submerged in the indoor tank and in front of the shark. This oxygen probe and temperature probe were configured to collect ambient dissolved oxygen saturation and temperature. Additionally, air stones were placed at the rear of the indoor tank behind the shark to keep the water saturated with oxygen. Each shark was weighed and measured after each trial.

### Individual Gill Measurements of Oxygen Extraction as Compared to Total Animal Measurements

Referring to FIG. 5, studies have found that gills two and three were the most efficient at extracting oxygen and had the least variable measurements. In this experiment, point measurements of oxygen extraction was taken at gill three for the subject six Sandbar Sharks while they were sedated in a static respirometry system, using the same method as described above. Unlike the previous experiment, the tank will be sealed with a fiberglass lid to prevent the exchange of oxygen. This will allow the concurrent estimation of SMR using traditional static respirometry, with the point measurements taken at the gill to validate the use of a single measurement as a successful proxy for full body oxygen consumption and by extension, the shark's full body metabolic rate.

### Swim Tunnel Respirometry

In this experiment, the subject sharks were subjected to a 950 L Loligo Swim Tunnel Respirometer on the subject six Sandbar Sharks. In particular, a narrow plastic tubing was sutured to the area right behind the gill and the oxygen and/or temperature probes were threaded through the tubing and positioned right inside the gill opening for the trials. To prevent visual disturbances that could lead to stress, the room the tank was situation was kept in the dark, and a curtain will be placed between the respirometer and the researcher. The animals were monitored via live video feed from a camera suspended above the tank. Prior to testing, sharks were fasted for 48 hours and acclimated to the tank for at least 10 hours overnight. Before the acclimation period, the narrow plastic tube was sutured to their gills (as shown in FIG. 5). Eight hours into the acclimation period, the shark was captured and the oxygen and/or temperature probes was threated through the tubing. While the shark is acclimating, the flush pump was turned on to continuously flush the system with fully oxygenated water.

After the acclimation period, the flush pump was turned off, the tank was sealed, and measurements of metabolic rate data and other data related to environmental conditions and motion of the animal, commenced. The sharks swam at four different speeds; 0.5, 0.7, 0.9, and, 1.1 body lengths per second (BL/s). They were allowed to draw down the oxygen in the tank until it reaches 80% saturation, during which time, measurements were taken every 60 seconds. When the oxygen reaches 80%, the tank was flushed until it is reoxygenated, resealed, and the shark was permitted to begin drawing down the oxygen again. This process was also repeated at least three times for each individual shark.

Once the trials are complete, the shark was removed from the tank and weighed. Different length measurements (precaudal length=PCL; fork length=FL; total length=TL) of the sharks were also obtained prior to returning the sharks into the tank. The respirometer was then re-sealed and background respiration data was collected for four hours.

### Analysis

Data from these experiments provided oxygen extraction at each individual gill for each shark and a comparison of point extraction to full body extraction. The experimental data also provides a validation of the inventive device and method. Metabolic rates are determined through the respR package in R. This package takes oxygen extraction at the gills, gill opening area, and exhalant flow rate, and processes it through the following equation:

$${O}_{2} = ( {C}_{i} {O}_{2} - {C}_{e} {O}_{2} ) FR$$

O₂ is the rate of oxygen consumption over time, CᵢO₂ and CₑO₂ are the incurrent and excurrent oxygen concentrations, and FR is the flowrate of water.

Additionally, three methods of acquiring whole body metabolic rate from individual gill oxygen consumption rates were tested to determine which can be used to more accurately represent a metabolic rate for the animal. The first method (method 1) incorporates gills specific flow and oxygen extraction rates estimated for each gill in respR to estimate whole animal metabolic rate by adding them, and multiplying by two to account for each set of gills. The second method (method 2) averages the flow rate across all gills and then the new exhalant extraction value generated with the average flow rate will be used in analysis in respR to determine oxygen consumption. Finally, the third method (method 3) calculates an average raw gill area and flow value data, and then use the new exhalant flow value generated in respR to determine oxygen consumption.

An ordinary least square regression was used to compare metabolic rates for each shark through all three methods of sampling: full body extraction determined by individual gill measurement, individual gill measurement in a sealed system vs whole body measurement, and flume respirometer. All was followed by a Tukey Honest Significant Difference (HSD) test to confirm significance.

Finally, as flow rate into and out of the gills is necessary to determine metabolic rate through gill extraction, video analysis was used during swim tunnel respirometry trials to determine the size of mouth gape as the shark is swimming at different speeds. From that, an equation and system for analyzed flow rate through the gills based on mouth gape and gill area may be developed for future use in field measurements.

### Further Progress

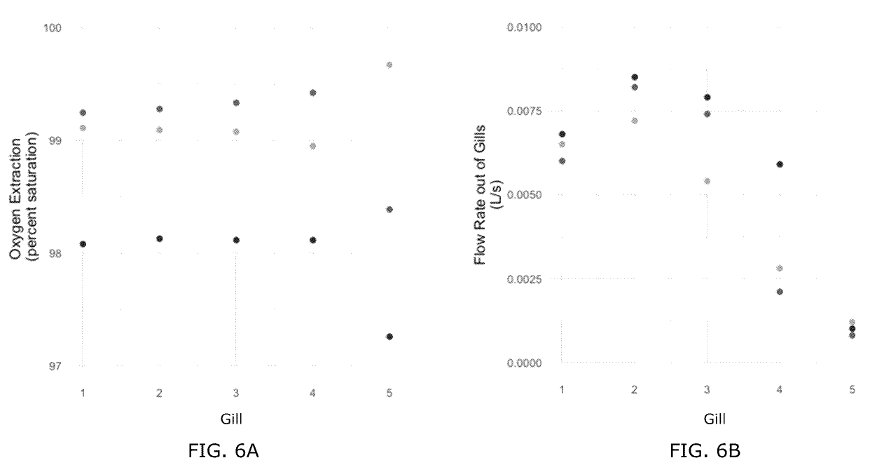

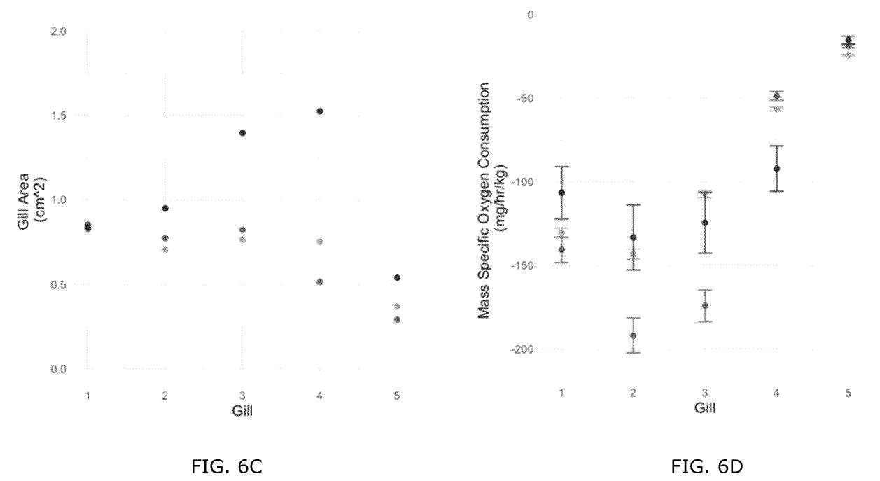

In summer/fall 2021, YOY Sandbar and Smooth Dogfish (Mustelus canis) sharks were collected by hook and line from Delaware Bay. They were maintained in a laboratory seawater system and these animals were used for developing and validating the method for calculating metabolic rate from gill exhalent water (deoxygenated seawater leaving the gills) for use in estimating FMR measurements. Determination of the flow volume out of the gills (FIG. 6B), and the area of gill openings (FIG. 6C) was measured and calculated. Specifically, videos of the gills of each shark were taken while respiring, and stills of the gills opened to their maximum extent were used to calculate the area of each gill opening in ImageJ. Sharks were placed in the tank as they would be for experiments and food dye was injected in to their mouths. During this process, video was taken, and the velocity of the dye leaving the gills was used to calculate exhalant flow in Tracker Software (Open Source Physics).

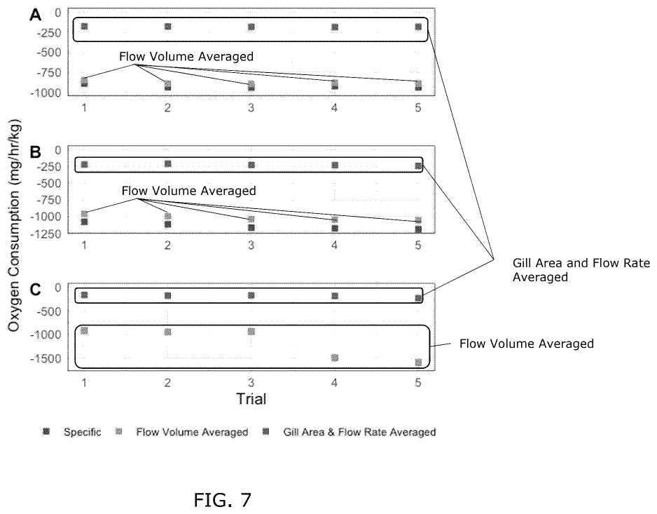

Initial data was collected for three individual Sandbar sharks for oxygen extraction (FIG. 6A) and consumption (FIG. 6D) at each individual gill at the specified flow rate (0.165 m/s). In particular, the data illustrated in FIGS. 6A-6D was used to compare different measures (flow rate, percent oxygen extraction, gill area and oxygen consumption (metabolic rate) from each gill for a set of subject animals. The data has been analyzed using the respR package discussed above. Additionally, the three methods for calculating whole body metabolic rate from the individual gill values (as described above) were tested (FIG. 7). In FIG. 7, the specific data points represent gills-specific flow and oxygen extraction rates estimated for each gill in respR package to estimate whole animal metabolic rate by adding them, and multiplying by two to account for each set of gills (in accordance with method 1 discussed above). The Flow Volume Averaqed data points represent average of flow rate across all gills and then the new exhalant extraction value generated with the average flow rate will be used in analysis in respR to determine oxygen consumption (in accordance with method 2 discussed above). The Gill Area & Flow Rate Averaqed data points represent averages raw gill area and flow value data, and then use the new exhalant flow value generated from that for analysis in respR to determine oxygen consumption (in accordance with method 3 discussed above).

### Example Two: Use of Static Respirometry, Swim Tunnel Respirometry, and the Subject Device and Method to Compare Metabolic Rate and Efficiency of Oxygen Extraction in Sandbars Across Sizes (YOY to Juvenile to Adult)

One purpose of this example is to validate the estimates of metabolic rate obtained via the inventive device and method of obtaining aquatic field metabolic rate data larger free-swimming animals through the use of a large static respirometer. After validation, preliminary field tests can ensure that the inventive device and method functions as it should in situ.

### Hypotheses

With increasing body size, an increase in whole animal metabolic rate and decrease in mass specific metabolic rate is expected. Oxygen consumption values—and by extension metabolic rate—collected with the inventive device and method in the flow through and static respirometers, as well as those collected in the field were expected to be comparable for animals of the same size.

### Methods

### PEL Respirometer

To determine oxygen consumption for animals larger than can be tested in the swim tunnel respirometer (up to 15 kg, Loligo Systems), a 20 ft diameter tank at the Pollution Ecology Laboratory at the University of Delaware's Lewes Marine Campus was modified to serve as an outdoor static respirometer. The tank was filled with water pumped directly from Delaware Bay and shaded to prevent thermal stress. A pump was placed along one side of the tank to aid in the circulation of water throughout. Two multi-parameter instruments, such as a multi-parameter instrument as manufactured and designed by YSI of Yellow Springs, Ohio, were placed on opposite sides of the tank, and used to collect data during the trials to ensure that an accurate representation of dissolved oxygen (DO) throughout the tank. The tank also contained a frame (e.g. a PVC frame) that supports the plastic sheeting used to seal the tank. When the sheeting is in place, a flexible PVC ring was used around the inside perimeter of the tank to create an impermeable seal and hold it in place during trials. The tank was filled to within a foot of the lip of the tank, and when trials are set to begin, it will be vented of bubbles, the PVC ring will be put in place, and the tank will be filled to the top with water. This layer of water on top of the plastic sheeting will help seal the sheeting in place and act as a buffer.

Sandbar Sharks between 100 to 175 cm in total length were collected from Delaware Bay. During transport to the large static respirometer, animals were placed in a livewell with aeration and water circulation. At that time, the inventive tag will be attached to the animal. Upon arrival at the dock, the sharks will be placed in a sling, and carried to the tank. The sharks weighed, measured (PCL, FL, TL) and placed in the respirometer. There, the shark will be allowed to acclimate for 48 hours. The tank was unsealed, and the shark was exposed to natural temperature and light regimes. Once the acclimation period is over, the tank was sealed and the shark will be undisturbed until the air saturation is drawn down to 80%. The tank was unsealed and re-oxygenated with air pumps, until saturation reaches 100% again. Throughout the course of the trial, Temperature (° C.) and DO (mg l⁻¹) measurements were taken every minute. Each shark was subjected to three closed periods within the respirometer. Once trials were completed, the sharks were returned to the Bay. The tank was resealed so background respiration data can be taken for four hours.

Before any trials, tests were conducted to determine if there is oxygen diffusion between the sealed tank and surrounding environment. The tank was bubbled with nitrogen. The respirometer was sealed with the pump and the YSI multi-parameter instruments in place, and temperature and dissolved oxygen measurements were taken every thirty seconds for three hours.

### Field Deployment Methods

Exemplary methods for deploying the subject device may include capturing Sandbar Sharks using handlines or hook and line in Delaware Bay. Upon capture, sharks may be weighed and measured (PCL, FL, TL) and then tagged with the subject device. Additionally, photographs may be taken of mouth gape, gill height and width for later analysis. The subject device may be fitted with a locating device, such as a smart position and temperature (SPOT) transmitting tag as manufactured and designed by Wildlife Computers of Redmond, Washington. The locating device may be releasably attached to the shark's dorsal or pectoral fin at two points using monofilament and a galvanic timed-release mechanism. This allows for eventual corrosion and subsequent release of the subject device after a predetermined or set number of days. Attachment of the subject device at the gill may be the same as with the swim tunnel respirometer trials discussed above (e.g. in objective one). The shark may be released, and the inventive device may be retrieved after it has fallen off. Upon corrosion and release of the inventive device, it is configured to float to the surface, where it will transmit its location to orbiting Argos satellites, and the inventive device can then be retrieved.

### Prospective Analysis

Data from the inventive device will be analyzed with the respR package to determine metabolic rates. As with objective one, ordinary least square regression will be used to compare metabolic rates for sharks between sharks in the static respirometer, the field and the laboratory.

Tri-axial accelerometer, and flowmeter data were analyzed to calculate overall dynamic body action (ODBA) and also assessed to determine changes in metabolic rate with different movement behaviors and environmental conditions throughout the duration of sampling. This determination of the cost of transport (COT) for different movement types could allow for a more accurate determination of energy expenditure in future bioenergetics models.

In addition to metabolic data, the inventive device may be configured to collect environmental data. Mixed effects models may be used to observe differences in metabolic rates based on dynamic changes in environment, with temperature and activity as fixed effects. Finally, metabolic data from the subject device during behavior that is equivalent with those from the flume respirometer (sustained swimming) may be compared to determine if a consistent mathematical transformation is possible to get laboratory data to more accurately represent field metabolic rates.

### Progress

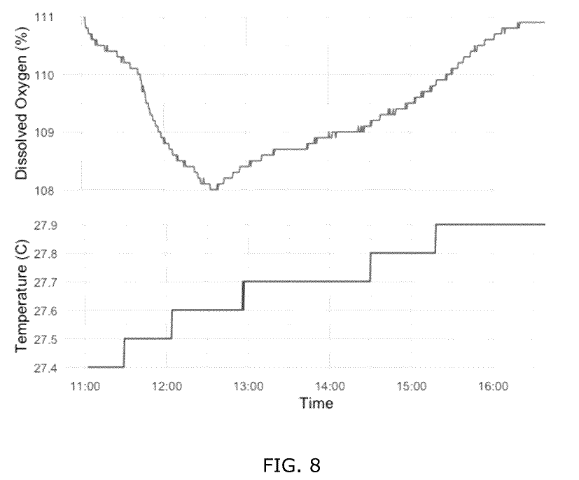

The static respirometer tank's PVC system has been constructed and all materials are in place as of summer 2021. To assess whether the plastic sheeting allows for the diffusion of oxygen from the surrounding environment, the tank was bubbled with pure nitrogen for 1.5 hours, which decreased air saturation levels. The pump and YSI were then put in place, and the system was sealed. Temperature (° C.) and dissolved oxygen (mg l⁻¹) measurements were taken every thirty seconds for three hours, during which time, the DO almost returned to its original levels (FIG. 8). This indicates that the system may need to be altered, likely with a smaller tank, which can be better sealed. Previous studies used a children's play pool with success, so it is likely implementing a smaller system, which can be more easily sealed, will remedy this issue in prospective experiments and studies.

During the summer of 2021, two sharks were caught and placed into the system as a test. Unfortunately, high temperatures forced the removal of the sharks and their return to the bay, so as to avoid any unnecessary mortality events. To avoid issues with temperature—both excess and variability—future prospective trials will be conducted in the evening between the hours of 1800 and 0900.

### Example Three: Compare Metabolic Rate and Efficiency of Oxygen Extraction Between a More Active Ram Ventilator (Sandbar Shark) and Less Active Species (Smooth Dogfish) that Exhibits Variable Respiration (Ram Ventilating and Buccal Pumping) in a Captive Controlled Setting

One purpose of this example is to compare metabolic rates of a more active species of shark and a less active species of shark in the laboratory setting.

### Hypotheses

The rate at which oxygen consumption increases with body mass is expected to be the same across species with different activity levels. Taking into account the effect of size, oxygen consumption—and thus metabolic rate—in an animal with an active ecological lifestyle (e.g. Sandbar Shark), is expected to be higher than one with a less active lifestyle (e.g. Smooth Dogfish).

### Methods

Methods described above with respect to Example One (Individual Gill Measurements of Oxygen Extraction, Individual Gill Measurements of Oxygen Extraction as Compared to Total Animal Measurements, and swim tunnel respirometry) were used to estimate metabolic rate of Smooth Dogfish in order to compare oxygen consumption and metabolic rate between Smooth Dogfish and Sandbar Sharks.

### Analysis

Analysis for the Smooth Dogfish was similar to that used for Sandbar Sharks in Example One, with the addition of a linear regression to compare Sandbar Shark and Smooth Dogfish metabolism for each treatment to observe differences in metabolic rates based on activity levels.

### Progress

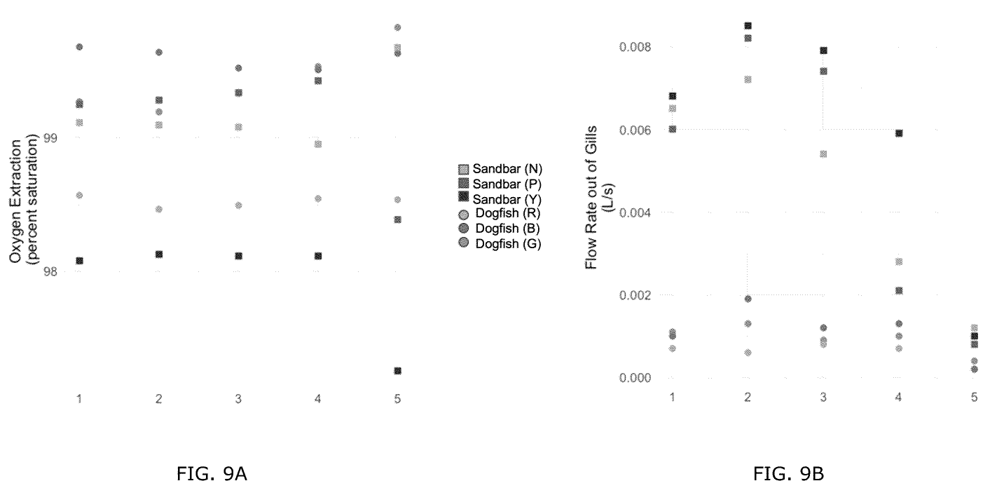

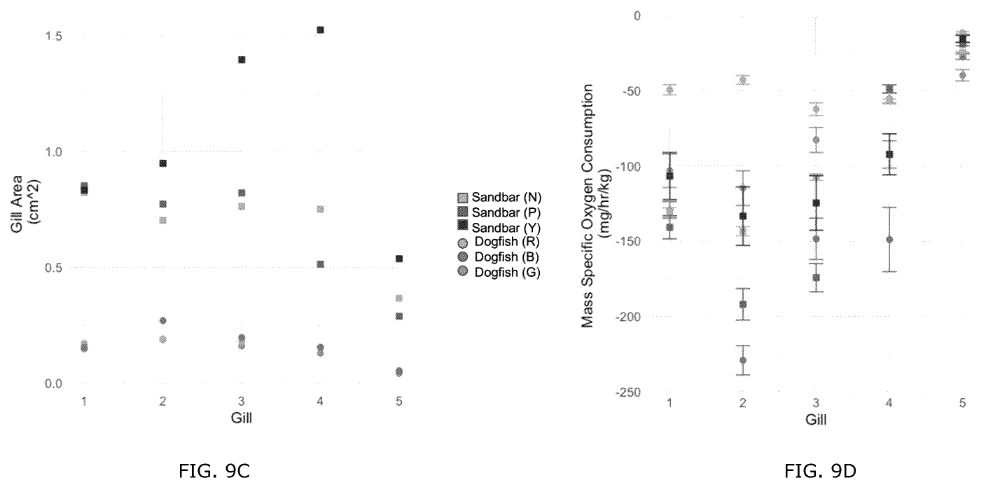

Initial data was collected for three Sandbar Sharks, and three Smooth Dogfish sharks for oxygen extraction and consumption at each individual gill at the specified flow rate. The data has been analyzed using the respR package in R, as discussed above, and the results are shown in FIGS. 9A-9D.

### Example Four: To Use the Subject Device to Compare Field Metabolic Rates Between an Active (Sandbar) and Inactive (Sand Tiger) Species, and to Determine how Changes in Metabolic Rate Based on Dynamic Changes in Activity and Environmental Conditions Differ from Captive Studies

One purpose of this example is to compare metabolic rates of a more active species and a less active species of shark in the field setting.

### Hypothesis

The subject device can accurately determine FMR in free swimming sharks. FMR in a species with an active ecological lifestyle will be higher than that of one with a less active lifestyle.

### Exemplary Methods

Sandbar and Sand Tiger (Carcharias taurus) sharks may be captured using handlines. The inventive device may be attached to the sharks following the method used in Example Two. The subject device may be deployed for two or three day stretches at a time. Upon recovery of the inventive device, data may be downloaded, the battery charged and the memory cleared, and it may be redeployed on a different shark.

### Analysis

Analysis is consistent with that described above for Example Two, with the addition of linear regressions to compare overall metabolic rates, and behavioral metabolic rates between Sandbar Sharks and Sand Tiger sharks.

### Example Five: Method to Estimate Whole Animal Metabolic Rate from Point Measurements of Gill Exhalant

To determine metabolic rate through oxygen consumption as well as oxygen extraction at a gill (e.g. third gill opening), dissolved oxygen content between gill inhalant water and gill exhalant water at the third gill opening is used to determine MO₂.

### Method

### Individual Gill Measurements of Oxygen Extraction

A prototype of an inventive gill exhalant oxygen probe, such as an oxygen probe as manufactured and designed by Pyroscience of Aachen, Germany, was inserted into the third gill of the subject shark(s) so that point measurements of oxygen extraction was taken at gill three for the subject sharks (e.g. Sandbar Sharks). Measurements of the dissolved oxygen levels in exhalant water (% air saturation) were collected for a predetermined duration, such as one minute. Another oxygen probe was submerged in the same water environment of the subject shark to collect ambient dissolved oxygen saturation. As discussed above in Example One, the water environment is a tank sealed with a fiberglass lid.

### Analysis

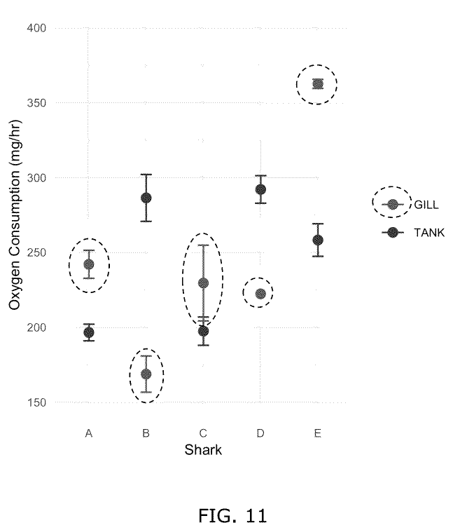

Collected data includes oxygen extraction at the opening of gill three for each shark as measured by the gill oxygen probe and the ambient oxygen probe. Referring now to FIG. 11, metabolic rates are determined by processing oxygen extraction at the gill opening area and exhalant flow rate through the following equation:

$${MO}_{2} = ( inhalant DO - exhalant DO ) FR$$

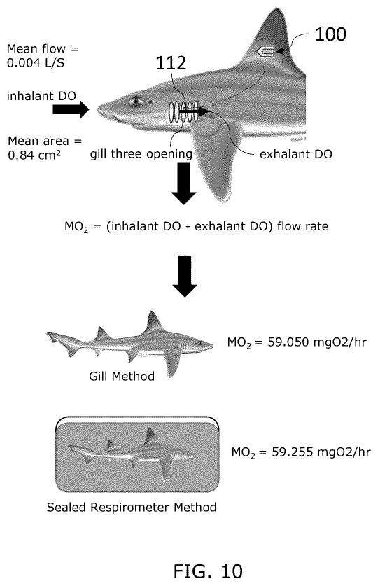

MO₂ is the rate of oxygen consumption over a predetermined duration by the at least one gill, inhalant DO is incurrent dissolved oxygen (DO) concentration, exhalant DO is excurrent dissolved oxygen (DO) concentration, and FR is the flowrate of water. In one experiment, as illustrated in FIG. 10, the whole-organism metabolic rate (MO₂) of a single Smooth Dogfish is calculated by determining an average gill opening area (e.g. 0.84 cm²), a flow rate (0.004 Liters/second (L/s)) and oxygen extraction (% saturation) at gill three for the subject shark.

With reference to FIG. 11, preliminary results from two trials each for five different Sandbar Sharks (A-E) were tested in the sealed water environment. The graph includes measurements (circle in dashed lines) representing the metabolic rate via oxygen consumption acquired from the inventive gill oxygen probe inserted inside the third gill opening (e.g. 5 mm within the third gill opening) of the subject sharks, as well as measurements (non-encircled in dashed lines) representing the metabolic rate via oxygen consumption acquired via an ambient oxygen probe placed in the sealed tank with the subject sharks. The values on the plot includes averages of two trials per single shark. A Mann-Whitney U test found no significant different between the tank and gill values n=5, p-value=0.8413). Accordingly, preliminary results suggest that that point measurements of oxygen extraction taken at gill three can provide a successful proxy for full body oxygen consumption and by extension, the shark's full body metabolic rate.

Although the invention is illustrated and described herein with reference to specific embodiments, the invention is not intended to be limited to the details shown. Rather, various modifications may be made in the details within the scope and range of equivalents of the claims and without departing from the invention.

1. A device configured to collect aquatic field metabolic rate data related to a water-breathing animal having at least one gill opening, the device comprising:
a plurality of sensors, the plurality of sensors comprising:
at least one oxygen probe configured to measure oxygen consumption or oxygen extraction data at the at least one gill opening;
a controller coupled to the plurality of sensors, the controller configured to perform one or more of the following operations in accordance with instructions stored in a digital memory:
(i) control one or more control settings of the plurality of sensors,
(ii) process and record metabolic rate data collected by the plurality of sensors, and
(iii) transmit metabolic rate data collected by the plurality of sensors to an external device;
wherein the device is configured to collect at least metabolic rate data when the animal is in a natural environment.

2. The device of claim 1, wherein oxygen consumption or oxygen extraction data comprises inhalant DO and exhalant DO at the at least one gill opening.

3. The device of claim 1, wherein the at least one oxygen probe comprises an ambient oxygen probe configured to measure dissolved oxygen levels in ambient water.

4. The device of claim 1, wherein the at least one oxygen probe comprises at least one gill exhalant oxygen probe configured to measure dissolved oxygen levels in exhalant water.

5. The device of claim 4, wherein the at least one gill exhalant oxygen probe is inserted within up to 5 mm into the at least one gill opening.

6. The device of claim 4, wherein the at least one gill exhalant oxygen probe is configured to measure dissolved oxygen levels in exhalant water for a predetermined duration or frequency.

7. The device of claim 1, further comprising an ambient temperature probe configured to measure temperature data of ambient water.

8. The device of claim 7, further comprising a gill exhalant oxygen probe configured to measure a temperature data of exhalant water.

9. The device of claim 1, further comprising a locating device configured to be releasably attached to the animal, the locating device being configured to track motion or location of the animal or the device and transmit the location of the animal or device to an external device.

10. The device of claim 1, further comprising a tri-axial accelerometer configured to collect, record, or transmit accelerometry data related to motion of the animal.

11. The device of claim 10, wherein the accelerometry data comprises swimming speed.

12. A method for determining a metabolic rate of a water-breathing animal having at least one gill and located in a water environment, the method comprising:
inserting a plurality of sensors in or adjacent the at least one gill opening of the animal, the plurality of sensors comprising at least one gill exhalant oxygen probe;
measuring aquatic field metabolic rate data of the animal via the plurality of sensors;
measuring ambient data in the water environment via at least one ambient oxygen probe and at least one ambient temperature probe; and
estimating a whole animal metabolic rate from one or more of the collected aquatic field metabolic rate data or ambient data.

13. The method of claim 12, wherein measuring aquatic field metabolic rate data comprises measuring inhalant DO and exhalant DO at the at least one gill opening, via the at least one gill oxygen probe.

14. The method of claim 13, wherein measuring ambient data comprises measuring ambient dissolved oxygen saturation, via the at least one ambient oxygen probe, in the water environment and measuring ambient temperature, via the at least one ambient temperature probe, in the water environment.

15. The method of claim 12, wherein the plurality of sensors further comprises at least one sensor configured to measure a flow rate (FR) of water through the at least one gill opening.

16. The method of claim 14, wherein estimating the whole animal metabolic rate comprises calculating a respective rate of oxygen consumption over a predetermined duration by the at least one gill (MO₂), which can be expressed by the following equation:
$${MO}_{2} = ( inhalant DO - exhalant DO ) FR$$

17. The method of claim 12, further comprising attaching a sutured tube to the at least one gill opening of the animal, wherein the at least one gill exhalant oxygen probe is inserted into or through the sutured tube.

18. The method of claim 12, further comprising releasably attaching a locating device to the animal, the locating device being configured to track motion or location of the animal or the device and transmit the location of the animal or device to an external device.

19. The method of claim 12, further comprising attaching a tri-axial accelerometer configured to collect, record, or transmit accelerometry data related to motion of the animal.
